(19) United States
(12) Patent Application Publication
(10) Pub. No.: US 2020/0165656 A1
(43) Pub. Date: May 28, 2020

(54) Systems & methods for providing an early indicator of true positive probability of a test sample

(71) Applicant: Stone Medical Corporation, Bettendorf, IA (US)

(72) Inventor: Benjamin Curtis Stone, Davenport, IA (US)

(21) Appl. No.: 16/695,748

(22) Filed: Nov. 26, 2019

Related U.S. Application Data
(60) Provisional application No. 62/771,693, filed on Nov. 27, 2018.

(51) Int. Cl. C12Q 1/02; G16H 10/40

(52) U.S. Cl. CPC C12Q 1/02; G16H 10/40

(57) ABSTRACT

The present disclosure provides illustrative embodiments of a system for tracking microbiology test samples with relatively lower rates of contamination in order to separate test results from samples with a higher positive predictive value (PPV) from those with a lower PPV in order to provide an early indicator of true positive probability and prevent delays in appropriate treatment of patients.

### CROSS REFERENCE TO RELATED APPLICATIONS

The present utility non-provisional patent application claims the filing benefit and priority of provisional U.S. Patent Application No. 62/771,693 filed on Nov. 27, 2018, which is incorporated by reference herein in its entirety.

### FIELD OF INVENTION

This present disclosure relates to systems for providing an early indicator of true positive probability of a microbiology test sample to improve treatment response time and accuracy.

### STATEMENT REGARDING FEDERALLY SPONSORED RESEARCH OR DEVELOPMENT

No federal funds were used to develop or create the invention disclosed and described in the patent application.

### REFERENCE TO SEQUENCE LISTING, A TABLE, OR A COMPUTER PROGRAM LISTING COMPACT DISK APPENDIX

Not Applicable

### BACKGROUND

Frequent contamination of microbiology test samples often limits the usefulness of the test by reducing the positive predictive value (PPV) below a threshold of reliability. It is not uncommon for body fluid cultures on blood, urine, and other samples to have contamination rates (false positive rates) that exceed the true positive rates. This lack of reliability can lead to significant delays in removal of key sources of infection, treatment of patients with antibiotics, failure to perform antimicrobial sensitivity testing on positive samples, and an even greater delay in treatment of patients with targeted narrow spectrum antibiotics. Delayed treatment, particularly removal of key sources of infection, can increase the mortality rate in cases of sepsis, urosepsis, septicemia, and other rapidly progressing diseases. Delays in delivery of targeted treatment can also expose the patient to longer general treatment with potentially severe side effects such as Clostridium difficile infections, vancomycin resistant Enterococcus infections, and damage to the liver and/or kidneys.

Microbiology tests such as blood cultures, urine cultures, and other body fluid cultures are often performed in multiple sets to help determine if a test result is a contaminated false positive or a true positive finding. Contamination is typically indicated when one test result is positive and several other test results from the same patient are negative. However, this determination is retrospective, often requiring several days and analysis of most of the test results before a specific sample can be identified as contaminated. This type of protocol has limited value towards treatment decisions in the earlier stages of sepsis when treatment may have the greatest impact on patient outcomes.

U.S. Pat. No. 6,913,580 discloses various methods and systems to reduce contamination of body fluid samples by diverting the initial body fluid flow prior to collection of a body fluid culture sample. While these methods are effective at reducing contamination and yield a high PPV, they may represent only a portion of the test results if compliance with usage is low or moderate. The ability to track and report test results from body fluid samples drawn with these types of methods along with the PPV, contamination rate, or another indicator of true positive probability can provide an early indicator that prevents delays in treatment of patients. The reduced time to general and targeted treatment can reduce rates of mortality, morbidity, and improve outcomes.

### SUMMARY OF THE PRESENT DISCLOSURE

The present disclosure describes systems for tracking microbiology test samples with relatively lower rates of contamination in order to separate test results from samples with a higher positive predictive value (PPV) from those with a lower PPV (wherein the PPV may be dependent at least in part on the system and/or method used to procure the body fluid sample). The PPV of a diagnostic test may be essential to healthcare providers when interpreting the results of the test. For example, a relatively high PPV can support decisions to treat patients without significant delay, while knowledge of a low PPV may lead healthcare workers to reconsider a patient's complete clinical case prior to providing treatment that may not be necessary, and/or may require retesting. Generally, an aspect of the present disclosure may include a system and/or method for separating diagnostic test results into groups based on the system and/or method of procuring the body fluid sample that was used for the diagnostic test, determining the PPV for those different groups, and reporting the PPV of the corresponding group to healthcare workers along with the diagnostic test results for a given patient. An illustrative embodiment of a system according to the present disclosure can prevent delay of appropriate treatment and simultaneously reduce the frequency of unnecessary treatment.

### BRIEF DESCRIPTION OF THE FIGURES

The accompanying drawings, which are incorporated in and constitute a part of this specification, illustrate embodiments and together with the description, serve to explain the principles of the methods and systems.

FIG. 1 is a depiction of first illustrative embodiment of a system & method for providing an early indicator of true positive probability of a test sample.

FIG. 2 is a depiction a second illustrative embodiment of a system & method for providing an early indicator of true positive probability of a test sample.

FIG. 3 provides an exploded view of an illustrative embodiment of a blood collection tube with a contact area and a tube seal cap.

FIG. 4 provides a perspective view of the illustrative embodiment of the blood collection tube shown in FIG. 3 with the tube seal cap in place to maintain sterility of the contact area.

FIG. 5 provides a perspective view of an illustrative embodiment of a needle holder having a marking device for tracking microbiology test samples.

FIG. 6 provides a perspective view of the illustrative embodiment of the needle holder from FIG. 5, wherein the needle holder is engaged with a device for collecting a microbiology test sample such that the marking device is in contact with the device for collecting a microbiology test sample.

### DETAILED DESCRIPTION OF THE PREFERRED AND ILLUSTRATIVE EMBODIMENTS

| Element | Element No. |
|---|---|
| Body fluid vessel | 1 |
| Body | 2 |
| Seal | 3 |
| Contact area | 4 |
| Indicator | 5 |
| Second vessel | 6 |
| Antimicrobial sensitivity test | 7 |
| Blood culture test results | 8 |
| Blood culture test data point | 9 |
| Indicator data point | 10 |
| Blood culture contamination rate data point | 11 |
| Blood culture report | 12 |
| Pre-marked blood collection tube | 13 |
| Hollow body | 14 |
| Seal | 15 |
| Contact area | 16 |
| Tube cap | 17 |
| Access window | 18 |
| Identifier | 19 |
| Tube cap seal | 20 |
| Nucleic acid preservation tube | 21 |
| Hollow body | 22 |
| Seal | 23 |
| Polymerase chain reaction test results | 24 |
| Identifier data point | 25 |
| Polymerase chain reaction test result data point | 26 |
| Positive predictive value data point | 27 |
| Polymerase chain reaction test report | 28 |
| Needle hub | 29 |
| Luer connector | 30 |
| Luer needle | 31 |
| Marking device | 32 |
| Device for collecting a microbiology test sample | 33 |

Before the present methods and apparatuses are disclosed and described, it is to be understood that the methods and apparatuses are not limited to specific methods, specific components, or to particular implementations. It is also to be understood that the terminology used herein is for the purpose of describing particular embodiments/aspects only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

“Aspect” when referring to a method, apparatus, and/or component thereof does not mean that limitation, functionality, component etc. referred to as an aspect is required, but rather that it is one part of a particular illustrative disclosure and not limiting to the scope of the method, apparatus, and/or component thereof unless so indicated in the following claims.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and apparatuses. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and apparatuses. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and apparatuses may be understood more readily by reference to the following detailed description of preferred aspects and the examples included therein and to the Figures and their previous and following description. Corresponding terms may be used interchangeably when referring to generalities of configuration and/or corresponding components, aspects, features, functionality, methods and/or materials of construction, etc. those terms.

It is to be understood that the disclosure is not limited in its application to the details of construction and the arrangements of components set forth in the following description or illustrated in the drawings. The present disclosure is capable of other embodiments and of being practiced or of being carried out in various ways. Also, it is to be understood that phraseology and terminology used herein with reference to device or element orientation (such as, for example, terms like “front”, “back”, “up”, “down”, “top”, “bottom”, and the like) are only used to simplify description, and do not alone indicate or imply that the device or element referred to must have a particular orientation. In addition, terms such as “first”, “second”, and “third” are used herein and in the appended claims for purposes of description and are not intended to indicate or imply relative importance or significance.

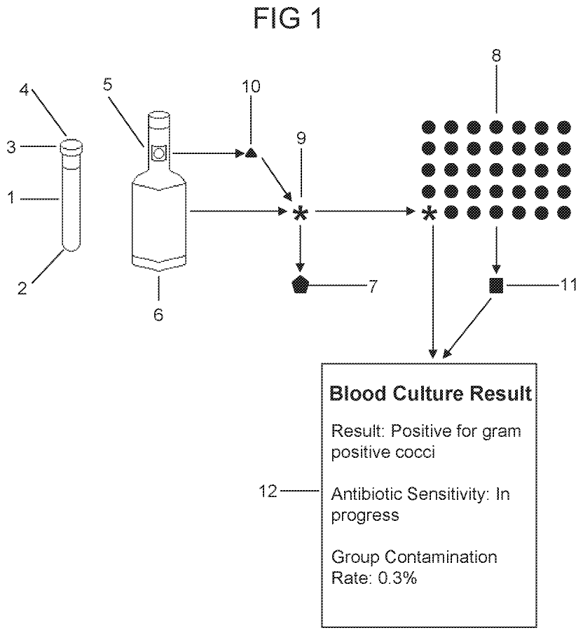

Referring now to the drawings, wherein like reference numerals designate identical or corresponding parts throughout the several views, FIG. 1 provides a schematic view of an illustrative embodiment of a system & method for providing an early indicator of true positive probability of a test sample. As shown, a first illustrative embodiment of a structure for collecting a body fluid sample may be configured as a body fluid vessel 1 (which may facilitate collection of a first body fluid specimen, and may also be referred to herein as a specimen container without limitation unless indicated in the following claims), which body fluid vessel 1 may be configured as a blood collection tube without limitation unless otherwise indicated in the following claims. The body fluid vessel 1 may be configured as an evacuated specimen tube comprising a hollow body 2 having an opening on one end, a seal 3 closing the open end of the hollow body 2, and a pressure within the body 2 that is lower than ambient atmospheric pressure to allow inflow of a body fluid sample when the seal 3 is pierced by a needle. The seal 3 may be configured with a contact area 4 positioned on an exterior-facing surface of the seal 3, wherein the contact area 4 may be subjected to environmental exposure and potential contaminants during use. A variety of body fluid vessels 1 (which may be configured as different types of blood collection tubes) may be used without departing from the scope of the present disclosure without limitation unless otherwise indicated in the following claims.

In a first illustrative embodiment, a sealed package may be used to maintain sterility of the contact area 4 of the body fluid vessel 1. The sealed package may be constructed from polypropylene, medical grade paper, or other packing materials well known in the art and/or materials suitable for the particular purpose without limitation unless otherwise indicated in the following claims. The sealed package may be hermitically sealed by heating, adhesives, or other sealing processes well known in the art or methods suitable for the particular application of the body fluid vessel 1. The sealed package may encompass only the body fluid vessel 1, or it may encompass the body fluid vessel 1 and other materials for collecting a microbiology test specimen without limitation unless indicated in the following claims.

Referring to still to FIG. 1, an illustrative embodiment of indicator 5 to identify a microbiology test sample may be configured as a decal. The indicator 5 may be configured with an adhesive surface capable of securing the indicator 5 to a surface. The indicator 5 may be attached to a non-adhesive backing material, and any suitable structure and/or method of engaging the indicator 5 with the desired surface may be used without limitation unless otherwise indicated in the following claims. In another illustrative embodiment, the indicator 5 may be configured as an electronic indicator, such as a field, checkbox, radio button, and/or other electronic indicia that a medical person (e.g., lab personnel, nurse, phlebotomist, etc.) may associate with an electronic record for that specific patient (such as an electronic medical record) to indicate the method used to collect the body fluid sample was a diversion method and/or other method without limitation unless otherwise indicated in the following claims.

In one illustrative embodiment, which may be a preferred embodiment for certain applications, a body fluid collection kit may be configured as a kit for collecting a blood culture sample, which may be configured as a sealed package (as discussed above regarding sterility of the contact area 4 of the body fluid vessel 1), wherein the sealed package may be configured to envelop the body fluid vessel 1 and indicator 5. The kit for collecting a blood culture may further comprise a variety of additional materials such as a needle hub, an IV extension, a blood culture bottle adaptor, an antiseptic, a set of gloves, a tourniquet, an IV dressing, a bandage, a blood culture bottle, the blood collection needle, and/or combinations thereof without limitation unless otherwise indicated in the following claims. A variety of designs and components are suitable for the kit for collecting a blood culture sample and this disclosure should not be construed as to limit the amount or type of additional materials unless otherwise indicated in the following claims. The kit for collecting a blood culture sample may or may not also be sterilized partially or entirely using ethylene oxide, gamma irradiation, or other suitable sterilization procedures.

With reference to FIG. 1, an illustrative embodiment of a second vessel 6 (which may be used for a microbiology test sample) may be configured as a blood culture bottle. The second vessel 6 may include a hollow body having an opening one end thereof, a seal, and a liquid medium. The hollow body of the second vessel 6 may be formed from glass or thermoplastic material. The seal of the second vessel 6 may be formed from a synthetic elastomer or natural rubber. The liquid medium may be a trypticase soy broth or other medium that supports the growth of microorganisms without limitation unless otherwise indicated in the following claims. A variety of materials are suitable for the construction of the hollow body and seal of the second vessel 6 and the scope of the present disclosure is not limited by the materials used in the construction thereof unless otherwise indicated in the following claims.

An illustrative embodiment of a body fluid access system may be comprised of a blood collection needle having a first end and a second end. The first end may be beveled for easier insertion through the patient's skin and into the vein. The second end may be beveled for easier passage through elastomeric seals. The blood collection needle may be constructed from steel and/or thermoplastic. A variety of materials are suitable for the construction of the body fluid access system and the scope of the present disclosure is not limited by the materials used in the construction thereof unless otherwise indicated in the following claims. The blood collection needle may further comprise a hollow tube disposed between the first end and the second end or a safety shield that can be configured to enclose the first end without limitation unless otherwise indicated in the following claims.

In a first illustrative method of use for collecting a blood sample, a user may employ the body fluid vessel 1 to remove potential contaminants from the body fluid access system (e.g., a blood collection needle) prior to collection of the blood culture sample into the second vessel 6. The collection method may further comprise a step of collecting a second blood culture into another second vessel 6. After collection of the desired blood culture sample(s) into the second vessel(s) 6, the indicator 5 may be attached to the surface of the appropriate second vessel 6.

In a first illustrative embodiment of a microbiology test, the microbiology test may be configured as a blood culture test comprising the steps of placing the second vessel 6 into an incubator and monitoring the second vessel 6 for a signal of microbial growth yielding a blood culture test result.

The blood culture test result may be a positive blood culture test result or a negative blood culture test result for microbial growth. Following a positive blood culture test result, the blood culture test may further comprise the steps of removing a subculture sample from the second vessel 6, performing a plate-based subculture test on the subculture sample, and performing an antimicrobial sensitivity test 7 on the subculture sample. Alternatively, the blood culture test may further comprise a test using polymerase chain reaction. A negative test result may be determined after a predetermined period of time in which the signal of microbial growth is not observed.

Referring to FIG. 1, a first illustrative embodiment of a classification method may classify a blood culture test result into a group of blood culture test results 8 yielded from blood culture tests performed on samples drawn using the same general steps described for the first illustrative embodiment of the collection method for a blood sample. Classification of the blood culture test result may be performed using a first classification procedure comprising the steps of: entering a blood culture test result data point 9 corresponding to the blood culture test result into a recordkeeping file; entering a indicator data point 10 indicating observation of the indicator 5 on the second vessel 6 into the recordkeeping file; associating the blood culture test result data point 9 and the indicator data point 10. The steps of the first illustrative embodiment of a classification method may be performed in a different sequence and this disclosure should not be construed as to limit the first classification method to a specific order unless otherwise indicated in the following claims.

A first illustrative embodiment of an indicator of true positive probability is a blood culture contamination rate. The blood culture contamination rate may be calculated using a first analysis procedure comprising the steps of: counting the number of blood culture test results from the group of blood culture test results 8 to yield a total number of blood culture test results; establishing a first criteria for identifying a false positive blood culture test result that indicates a contaminated blood culture sample; applying the first criteria to the group of blood culture test results 8; counting the number of false positive blood culture test results; dividing the number of false positive blood culture test results by the total number of blood culture test results to yield the blood culture contamination rate for the group of blood culture test results; generating a blood culture contamination rate data point 11 corresponding to the blood culture contamination rate; entering the contamination rate data point 11 into the recordkeeping file.

The procedure for determining a blood culture contamination rate may be performed by a manual calculation, electronic calculation, or a combination of manual and electronic methods. The steps to determine a blood culture contamination rate may be performed in a different sequence than indicated above and this disclosure should not be construed as to limit the procedure for determining a blood culture contamination rate to the order described unless otherwise indicated in the following claims.

Referring still to FIG. 1, a first illustrative embodiment of a report is a blood culture report 12. The blood culture report 12 may be generated using a first illustrative embodiment of a reporting procedure comprising the steps of: displaying the blood culture test data point 9 on the report 12; associating the indicator data point 10 to the blood culture test result data point 9; displaying the blood culture contamination rate data point 11 on the report 12. The steps of the first illustrative embodiment of a reporting procedure may be performed in a different sequence and this disclosure should not be construed as to limit the first reporting procedure to the order described unless otherwise indicated in the following claims.

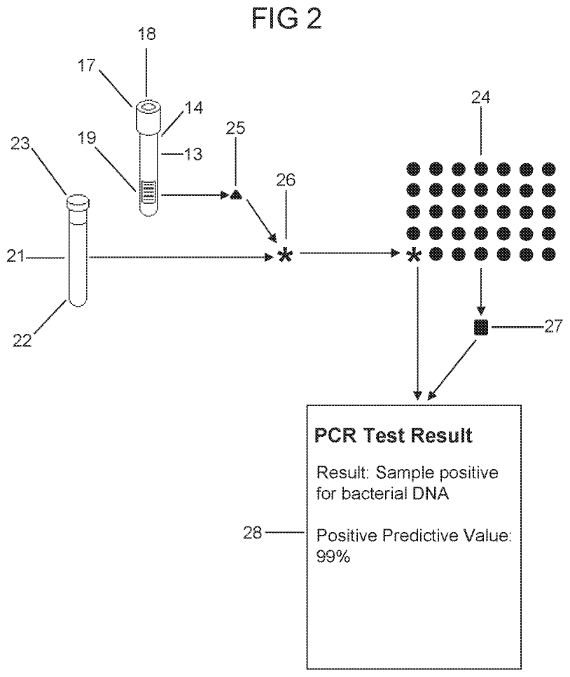

Referring now to FIG. 2, a second illustrative embodiment of a system & method for providing an early indicator of true positive probability of a test sample is shown therein, wherein the second illustrative embodiment uses more advanced materials and techniques compared to the first illustrative embodiment thereof described above and depicted in FIG. 1. The second illustrative embodiment may be preferable to achieve greater system consistency.

With continued reference to FIG. 2, a second illustrative embodiment of a structure for collecting a first body fluid specimen may be configured a pre-marked blood collection tube 13 (which may be another type of specimen container according to the present disclosure) comprising a hollow body 14 having an opening at one end thereof, a seal 15 closing the open end of the hollow body 14, a contact area 16, a tube cap 17 having an access window 18, and a scannable identifier 19 attached to the hollow body 14. The scannable identifier 19 may be a line type barcode, a matrix barcode, a radiofrequency tag, QR code, or another type of scannable material or device. A variety of materials may be used for the scannable identifier 19 and this disclosure should not be construed as to limit the scannable identifier to those described, but instead the scope of the present disclosure extends to any suitable structure and/or method for a scannable identifier without limitation unless otherwise indicated in the following claims. The scannable identifier 19 may further comprise an adhesive for attachment to the hollow body 14.

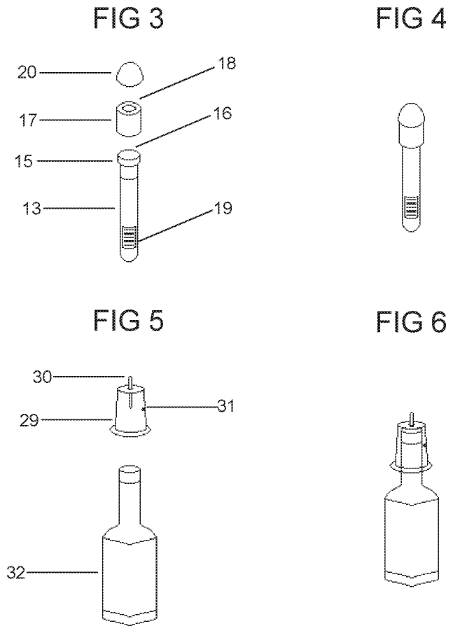

Referring now to FIG. 3, a second illustrative embodiment of a system and method to maintain sterility of the contact area 16 is a tube cap seal 20. As shown in FIG. 4, the tube cap seal 20 may be situated over the contact area 16 prior to or after sterilization in order to maintain sterility of the contact area 16. The tube cap seal 20 depicted in FIG. 3 may be configured to be reversibly attached to the access window 18 in the tube cap 17. In other embodiments, a tube cap seal 20 may be reversibly attached to the hollow body 14 or to the seal 15. The tube cap seal 20 may be attached by adhesive, screw threading, friction, or other structures and/or methods of attachment without limitation unless otherwise indicated in the following claims. The tube cap seal 20 may be constructed from injection molded thermoplastic, extruded thermoplastic film, combinations thereof, or other suitable materials. A variety of configurations are suitable for the tube cap seal 20 and this disclosure should not be construed as to limit configuration or design of the tube cap seal 20 to those described and/or depicted herein unless otherwise indicated in the following claims.

Another illustrative embodiment of a structure for collecting a body fluid sample may be configured to collect a microbiology test sample, wherein the structure may be configured as a nucleic acid preservation tube 21 comprising a hollow body 22 having an opening at one end thereof, a seal 23 closing the open end of the hollow body 22, and a preservative for nucleic acids. The hollow body 22 can be formed from glass or thermoplastic materials. The seal 23 may be formed from a synthetic elastomer or natural rubber. The preservative may comprise an anticoagulant such as ethylenediamine tetraacetic acid or sodium citrate, a nucleic acid stabilizing agent, or a combination of additives and solutions. A variety of preservatives and additives are suitable for the nucleic acid preservation tube 21 and this disclosure should not be construed as to limit the types of preservatives and additive used to those described and the scope of the present disclosure extends to all suitable preservatives unless otherwise indicated in the following claims.

Another illustrative embodiment of a body fluid access system to access a patient's body fluid may be comprised of an intravenous needle assembly, wherein the assembly comprises an intravenous needle comprising cylindrical body and a first end, an intravenous catheter comprising a first female luer fitting and a hollow body, and an access device comprising a first male luer fitting and a second end. The intravenous device assembly may be configured such that the cylindrical body is at least partially situated inside the intravenous catheter prior to insertion into the patient's body fluid vessel. The first end may be beveled for easier insertion through the patient's skin and into the body fluid vessel. The second end may be beveled for easier passage through elastomeric seals. The intravenous needle may be constructed from steel and thermoplastic. The intravenous catheter may be constructed from thermoplastic. The access device may be constructed from thermoplastic and stainless steel. A variety of materials are suitable for construction of the intravenous needle assembly and this disclosure should not be construed as to the limit the materials to those described unless otherwise indicated in the following claims. The intravenous needle assembly may further comprise an extension device comprising a second male luer fitting, a second female luer fitting, and a hollow extension tube disposed between the second male luer fitting and the second female luer fitting.

Generally, the second illustrative embodiment of a collection method may use the pre-marked blood collection tube 13 to remove potential contaminants from the intravenous catheter prior to collection of the microbiology test sample into the nucleic acid preservation tube 21. The collection method may comprises the steps of: collecting a body fluid sample by piercing through a patient's skin using the first end of the intravenous needle assembly; removing the intravenous needle from the intravenous catheter such that a portion of the hollow body of the intravenous catheter remains in a patient's body fluid vessel; attaching the first male luer fitting of the access device to the first female luer fitting of the intravenous catheter; piercing through the seal 15 of the pre-marked blood collection tube 13 using the second end of the access device such that potential contaminants are drawn into the pre-marked blood collection tube 13; collecting a microbiology test sample by piercing though the seal 23 of the nucleic acid preservation tube using the second end of the access device.

A second illustrative embodiment of a microbiology test is a polymerase chain reaction test comprising the steps of: addition of a lysis reagent to the microbiology test sample; addition of a deoxyribonucleic acid degradation agent to the microbiology test sample; centrifugation of the microbiology test sample and removal of a supernatant; addition of a cell wall lysis reagent to the microbiology test sample; transfer of the microbiology test sample to a purification column; addition of a washing solution to the purification column, addition of an eluting solution to the microbiology test sample; addition of a mixture comprising a primer set specific for microorganism-related nucleic acid polymer sequences, a polymerase, and a plurality of nucleotides to the microbiology test sample; placement of the microbiology test sample in a polymerase chain reaction chamber; cycling the microbiology test sample through a denaturing temperature phase, an annealing temperature phase, and an elongation temperature phase; addition of a reporter to the microbiology test sample; observation of the microbiology test sample for the reporter to yield a polymerase chain reaction test result.

The polymerase chain reaction test result may be a positive test result or a negative test result for the reporter indicating the presence of microorganism-related nucleic acid polymer sequences. A variety of materials, reagents, solutions, and steps are suitable for the polymerase chain reaction test and this disclosure should not be construed as to the limit the materials, reagents, solutions, and steps (and/or order thereof) to those described unless otherwise indicated in the following claims.

Referring to FIG. 2, the polymerase chain reaction test result may be classified into a group of polymerase chain reaction test results 24 yielded from polymerase chain reaction tests performed on body fluid samples drawn using the same general steps described for the second embodiment of the collection method. Classification of the polymerase chain reaction test result may be performed using a second classification procedure comprising the steps of: scanning the scannable identifier 19 using a scanner; generating an identifier data point 25 indicating presence of the pre-marked blood collection tube 13 and nucleic acid preservation tube 21; entering a polymerase chain reaction test result data point 26 corresponding to the polymerase chain reaction test result into a computer based electronic recordkeeping system; associating the identifier data point 25 and the polymerase chain reaction test result data point 26. The steps of the second classification procedure may be performed in a different sequence and this disclosure should not be construed as to limit the second classification procedure to the order described unless otherwise indicated in the following claims. Scanning of the scannable identifier 19 may occur during receiving of the pre-marked blood collection tube 13 and prior to the polymerase chain reaction test.

A second illustrative embodiment of an indicator of true positive probability is a positive predictive value. The positive predictive value may be calculated using an analysis procedure comprising the steps of: counting the number polymerase chain reaction test results 24 from the group of polymerase chain reaction test results 24 to yield a total number of polymerase chain reaction test results 24; counting the number of positive test results from the group of polymerase chain reaction test results 24; establishing a second criteria for identifying a false positive polymerase chain reaction test result that indicates a contaminated body fluid sample; applying the second criteria to the group of polymerase chain reaction test results 24; counting the number of false positive polymerase chain reaction test results; dividing the number of false positive polymerase chain reaction test results by the number of positive test results to yield the positive predictive value for the group of polymerase chain reaction test results 24; generating a positive predictive value data point 27 corresponding to the positive predictive value.

The procedure for determining a positive predictive value described above may be performed by a manual calculation, computer-based electronic software, or a combination of manual and electronic methods. The steps to determine a positive predictive value may be performed in a different sequence than indicated above and this disclosure should not be construed as to limit the procedure for determining a blood culture contamination rate to the order described unless otherwise indicated in the following claims.

Referring to FIG. 2, a second illustrative embodiment of a report may be configured as a polymerase chain reaction test report 28. The polymerase chain reaction test report 28 may be generated using a second reporting procedure comprising the steps of: displaying the polymerase chain reaction test data point 25 on the polymerase chain reaction test report 28; associating the identifier data point 25 to the polymerase chain reaction test result data point 26; displaying the positive predictive value data point on the polymerase chain reaction test report 28. The polymerase chain reaction test report 28 may remain in electronic form or be converted to another medium. The steps for generating and/or constructing a polymerase chain reaction test report 28 may be performed in a different sequence than indicated above and this disclosure should not be construed as to limit the method of generating a polymerase chain reaction test report 28 to the order described unless otherwise indicated in the following claims.

The second illustrative embodiment of the system & method for providing an early indicator of true positive probability of a test sample may further comprise a compliance feedback loop comprising the steps of: entering a blood collector data point into the electronic recordkeeping system; associating the blood collector data point to the polymerase chain reaction test result data point 26; generating a compliance report comprising the blood collector data point and polymerase chain reaction test data point 26. The compliance feedback loop is used to identify personnel using the collection method. Comparison of the compliance report to data for polymerase chain reaction tests collected by other methods may be used to identify personnel not using the collection method and targeting identified personnel for corrections.

Referring to FIG. 5, another embodiment of a system to identify a microbiology test sample is shown therein, wherein the system comprises a needle hub 29 having a luer connector 30, a luer needle 31, and a marking device 32. As illustrated in FIG. 6, the marking device 32 may be configured to contact an area on a device for collecting a microbiology test sample 33 leaving a mark for tracking. The needle hub 30 may be constructed from injection molded thermoplastics and connect to the luer connector 30 by screw threading. The luer needle 31 may be constructed from injection molded thermoplastics, steel, and an elastomer. The luer needle 31 may be beveled to allow for penetration of elastomeric seals. The marking device 32 may be comprised of a filler material at least partially saturated with an oil-based dye. The marking device 32 may further comprise a cover material that reduces evaporation of an oil-based dye and is removable by advancement of the device for collecting a microbiology test sample 33 into the needle hub 29 as shown in FIG. 6. The needle hub 29, the luer connector 30, the luer needle 31, and the marking device 32 may be constructed from other suitable materials and assembled in a variety of configurations and the scope of the present disclosure extends to all suitable variations without limitation unless otherwise indicated in the following claims.

In a third embodiment of a system & method for providing an early indicator of true positive probability of a test sample, the system uses the steps of: collecting a microbiology test sample into a specimen vessel using a collection method; providing an identifier of the collection method; performing a microbiology test using the microbiology test sample to yield a microbiology test result; entering the microbiology test result into a recordkeeping system; calculating a contamination rate for a group of test results comprising test results from microbiology tests performed on microbiology test samples collected using said collection method; generating an indicator of true positive probability based on the contamination rate; using the identifier of the collection method to classify the microbiology test result into the group of test results; generating a report comprising the microbiology test result; and providing the indicator of true positive probability.

A first embodiment of the specimen vessel is the blood culture bottle described in the first illustrative embodiment of the system. A second embodiment of the specimen vessel may be a urine specimen container of the kind having a hollow body and a lid formed of materials including but not limited to thermoplastic. This type of urine specimen container is well known in the prior art. A third embodiment of the specimen vessel is the nucleic acid preservation tube described in the second illustrative embodiment of the system. A fourth embodiment of the specimen vessel is a specimen tube of the kind having a hollow body and well known in the art. The specimen vessel may be another structure capable of holding a body fluid sample such as a container, tube, plate, dish, swab, or vial and the scope of the present disclosure should not be construed as to limit the specimen vessel to the types, configurations, or material of construction described unless otherwise indicated in the following claims.

The microbiology test sample may be a blood sample, urine sample, peritoneal fluid sample, lymph fluid sample, lacrimal fluid sample, saliva sample, or cerebrospinal fluid sample without limitation unless otherwise indicated in the following claims. A variety of other body fluids or combinations of body fluids may be used as the microbiology test sample and the scope of the present disclosure should not be construed as to limit the microbiology test sample to those listed or described unless otherwise indicated in the following claims.

A first embodiment of the collection method uses the steps of establishing blood flow into a blood collection needle of the kind well known in the art of blood collection by piercing through the skin of a patient using the blood collection needle, collecting a blood sample through the blood collection needle into a body fluid vessel of the kind described in the first illustrative embodiment of the system, and collecting a microbiology test sample into the blood culture bottle through the blood collection needle. A second embodiment of the collection method uses the steps of establishing a urine output stream from a patient and placing the urine specimen container in the urine output stream such that a urine sample is collected into the urine specimen container. A third embodiment of the collection method uses a collection device of the kind comprising a chamber, a first end, and a second end. The first end may further comprise a first needle and the second end may further comprise a second needle. Such collection devices are known in the art. The third embodiment of the collection method uses the steps of establishing body fluid flow through the first end, collecting a body fluid sample into the chamber through the first end, and collecting a microbiology test sample into the specimen vessel through the second end. A fourth embodiment of the collection method uses a urine collection device of the kind having an inlet, a first outlet, and a second outlet. Such urine collection devices are known in the art. The fourth embodiment of the collection method uses the steps of establishing a urine stream into the inlet, establishing a first urine output stream from the first outlet, establishing a second urine output stream from the second outlet, and collecting the urine sample into the urine specimen container from the second urine output stream. A fifth embodiment of the collection method uses a collection needle of the kind well known in the art of body fluid collection. The fifth embodiment of the collection method uses the steps of inserting the collection needle through a patient's skin and collecting a body fluid sample into the specimen vessel. A variety of other body fluid collection methods such as wound drainage, sampling through a urine catheter, intravenous catheter sampling, body cavity drainage sampling, and collection methods that may reduce the risk of contamination may be used as the collection method and the scope of the present disclosure should not be construed as to limit the collection method to those listed or described unless otherwise indicated in the following claims.

A first embodiment of the identifier of the collection method may be the indicator 5 (which may be configured as a decal) described in the first illustrative embodiment of the system. A second embodiment of the identifier of the collection method may be the scannable identifier described in the second illustrative embodiment of the system. A third embodiment of the identifier of the collection method may be the electronic indicator described in the first illustrative embodiment of the system in which data is entered directly into the recordkeeping system. A variety of other identifiers such as stamps, markings, textural materials, color change indicators, recorded audio notes, checklists, and written notes may be used as the identifier of the collection method and the scope of the present disclosure should not be construed as to limit the identifier of the collection method to those listed or described unless otherwise indicated in the following claims.

A first embodiment of the microbiology test may be a blood culture test as described in the first illustrative embodiment of the system. A second embodiment of the microbiology test may be a polymerase chain reaction test as described in the second illustrative embodiment of the system. A third embodiment of the microbiology test may be a urine culture test of the kind using agar-based plates streaked with a portion of the urine sample. Such urine culture tests are well known in the art of microbiology. A fourth embodiment of the microbiology test is a body fluid plate culture of the kind using agar-based plates and is well known in the art of microbiology. A fifth embodiment of the microbiology test is a matrix assisted laser desorption ionization time of flight mass spectroscopy test on a mass spectroscopy sample prepared from the microbiology test sample and is well known in the art of microbiology. A variety of other microbiology tests such as slant cultures, swab cultures, or other polymerase chain reaction tests may be used as the microbiology test to yield a microbiology test result and the scope of the present disclosure should not be construed as to limit the microbiology test to those listed or described unless otherwise indicated in the following claims.

A first embodiment of the recordkeeping system may be a manual records system in which data is primarily entered, distributed, copied, transferred, and stored in hardcopy form. Such manual records systems are well known in the art of healthcare recordkeeping. A second embodiment of the recordkeeping system may be an electronic medical records system in which data is primarily entered, distributed, copied, transferred, and stored in electronic form. Such electronic medical records systems are well known in the art of healthcare recordkeeping. A variety of recordkeeping systems and mixed recordkeeping systems using audio recording and other data recording methods in combination with electronic data and hardcopies may be used as the recordkeeping system and the scope of the present disclosure should not be construed as to limit the recordkeeping system to those listed and described unless otherwise indicated in the following claims.

The contamination rate may be calculated using an analysis procedure. A first embodiment of the analysis procedure is the first analysis procedure described in the first illustrative embodiment of the system using a first criteria applied to determine which test results are contaminants. The first criteria may be based on clinical data including but not limited to patient temperature, proportion of positive similar microbiology test results, number of positive microbiology test results, blood pressure, anion gap, white blood cell count, microbiology test results from alternate sources, lactic acid results, procalcitonin results, erythrocyte sedimentation rate, c-reactive protein results, microorganism species or type, gram testing results, microbial growth pattern, microbial growth rate, time to positive, subculture results, antimicrobial sensitivity test results, nucleic acid testing, polymerase chain reaction testing, other diagnostic tests, or diagnosis of a patient. Such clinical data may be used individually or in various combinations for the first criteria or any additional criteria used for subsequent analysis. A second embodiment of the analysis procedure may use the first criteria for a first analysis of the group of test results followed by use of a second criteria for a second analysis of the group of test results. The analysis procedure may use any number of criteria and may be applied to a portion or the entirety of the group of test results using manual methods, automated methods, or a combination of methods. The analysis procedure may also be performed once on the group of test results to calculate the contamination rate, repeated with new test results added to the group of test results, or recalculated for a group of test results generated within a selected time period. The scope of the present disclosure should not be construed as to limit the analysis procedure to those listed and described unless otherwise indicated in the following claims.

A first embodiment of the indicator of true positive probability may be the blood culture contamination rate calculated using the first analysis procedure described in the first illustrative embodiment of the system. A second embodiment of the indicator of true positive probability may be the positive predictive value calculated using the analysis procedure described in the second illustrative embodiment of the system. A third embodiment of the indicator of true positive probability may be a description indicating low, medium, or high probability that the microbiology test result is true positive based on the positive predictive value described in the second illustrative embodiment of the system. A fourth embodiment of the indicator of true positive probability may be a set of color-coded indicators such as red, yellow, and green each indicating a range of positive predictive values. A fifth embodiment of the indicator of true positive probability may be a group name. A sixth embodiment of the indicator of true positive probability may be a numeric code. A seventh embodiment of the indicator of true positive probability may be a reference to a database. An eighth indicator of true positive probability may be a reference to a related report. A variety of indicators of true positive probability such as graphs, figures, audio files, highlights, notes, text colors, tags, or codes may be used as the indicator of true positive probability and the scope of the present disclosure should not be construed as to limit the indicator of true positive probability to those described unless otherwise indicated in the following claims.

Classification of the microbiology test result into a group of test results may be performed using a classification procedure. A first embodiment of the classification procedure may be the first classification procedure described in the first illustrative embodiment of the system. A second embodiment of the classification procedure may be the second classification procedure described in the second illustrative embodiment of the system. A third embodiment of the classification procedure may use the steps of observing the collection method, entering the electronic indicator into the recordkeeping system, and associating the electronic indicator with the microbiology test result. A fourth embodiment of the classification procedure may use the steps of applying the identifier of the collection method to the specimen vessel, observing the identifier of the collection method, and entering the indicator of true positive probability into the report. A variety of classification procedures using different steps, sequences, methods of observation, methods of recording, and methods of transmitting information may be used as the classification procedure and the scope of the present disclosure should not be construed as to limit the classification procedure to those listed and described unless otherwise indicated in the following claims.

The report may be generated using a reporting procedure. A first embodiment of the reporting procedure comprises the steps of initiating the report, adding the microbiology test result to the report, and adding the indicator of true positive probability to the report. A second embodiment of the reporting procedure comprises the steps of initiating a microbiology report and adding the microbiology test result to the report. The report may be generated in various forms including but not limited to electronic, hardcopy, audio, or a combination of forms and the present disclosure should not be construed as to limit the report to those described. The reporting procedure may use different steps, sequences, additional steps, methods of transmission, and methods of storage and the scope of the present disclosure should not be construed as to limit the reporting procedure to those listed and described unless otherwise indicated in the following claims.

The indicator of true positive probability may be added to the report as described for the first embodiment of the reporting procedure or may be provided by a less direct route when using the second embodiment of the reporting procedure such as a reference to an additional report, link to a database, a reference code, audio file, or other forms. The indicator of true positive probability may be provided using different steps, sequence, additional steps, methods of transmission, and methods of storage and the scope of the present disclosure should not be construed as to limit the method of providing the indicator of true positive probability to those listed and described unless otherwise indicated in the following claims.

The present invention may include steps, procedures, materials, devices, apparatuses, and other elements from any preferred embodiment described or anticipated embodiments in various combinations and this disclosure should not be construed as to limit the present invention to the preferred embodiments described unless otherwise indicated in the following claims.

Although the methods described and disclosed herein may be configured to utilize a body fluid comprised of blood and the various illustrative embodiments described and pictured herein are configured for a body fluid comprised of blood, the scope of the present disclosure, any discrete process step and/or parameters therefor, and/or any apparatus for use therewith is not so limited and extends to any beneficial and/or advantageous use thereof without limitation unless so indicated in the following claims (e.g., saliva, urine, etc.).

The materials used to construct the apparatuses and/or components thereof for a specific process will vary depending on the specific application thereof, but it is contemplated that polymers, synthetic materials, metals, metal alloys, natural materials, and/or combinations thereof may be especially useful in some applications. Accordingly, the above-referenced elements may be constructed of any material known to those skilled in the art or later developed, which material is appropriate for the specific application of the present disclosure without departing from the spirit and scope of the present disclosure unless so indicated in the following claims.

Having described preferred aspects of the various processes, apparatuses, and products made thereby, other features of the present disclosure will undoubtedly occur to those versed in the art, as will numerous modifications and alterations in the embodiments and/or aspects as illustrated herein, all of which may be achieved without departing from the spirit and scope of the present disclosure. Accordingly, the methods and embodiments pictured and described herein are for illustrative purposes only, and the scope of the present disclosure extends to all processes, apparatuses, and/or structures for providing the various benefits and/or features of the present disclosure unless so indicated in the following claims.

While the chemical process, process steps, components thereof, apparatuses therefor, products made thereby, and impregnated substrates according to the present disclosure have been described in connection with preferred aspects and specific examples, it is not intended that the scope be limited to the particular embodiments and/or aspects set forth, as the embodiments and/or aspects herein are intended in all respects to be illustrative rather than restrictive. Accordingly, the processes and embodiments pictured and described herein are no way limiting to the scope of the present disclosure unless so stated in the following claims.

Although several figures are drawn to accurate scale, any dimensions provided herein are for illustrative purposes only and in no way limit the scope of the present disclosure unless so indicated in the following claims. It should be noted that the welding processes, apparatuses and/or equipment therefor, and/or impregnated and reacted upon substrates produced thereby are not limited to the specific embodiments pictured and described herein, but rather the scope of the inventive features according to the present disclosure is defined by the claims herein. Modifications and alterations from the described embodiments will occur to those skilled in the art without departure from the spirit and scope of the present disclosure.

Any of the various features, components, functionalities, advantages, aspects, configurations, process steps, process parameters, etc. of a chemical process, a process step, a substrate, and/or a impregnated and reacted substrate, may be used alone or in combination with one another depending on the compatibility of the features, components, functionalities, advantages, aspects, configurations, process steps, process parameters, etc. Accordingly, an infinite number of variations of the present disclosure exist. Modifications and/or substitutions of one feature, component, functionality, aspect, configuration, process step, process parameter, etc. for another in no way limit the scope of the present disclosure unless so indicated in the following claims.

It is understood that the present disclosure extends to all alternative combinations of one or more of the individual features mentioned, evident from the text and/or drawings, and/or inherently disclosed. All of these different combinations constitute various alternative aspects of the present disclosure and/or components thereof. The embodiments described herein explain the best modes known for practicing the apparatuses, methods, and/or components disclosed herein and will enable others skilled in the art to utilize the same. The claims are to be construed to include alternative embodiments to the extent permitted by the prior art.

Unless otherwise expressly stated in the claims, it is in no way intended that any process or method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including but not limited to: matters of logic with respect to arrangement of steps or operational flow; plain meaning derived from grammatical organization or punctuation; the number or type of embodiments described in the specification.

What is claimed is:

1) A system for providing an early indicator of true positive probability of a test sample comprising the steps of:
a. collecting a microbiology test sample into a specimen vessel using a collection method;
b. providing an identifier of said collection method;
c. performing a microbiology test using said microbiology test sample to yield a microbiology test result;
d. entering said microbiology test result into a recordkeeping system;
e. calculating a contamination rate for a group of test results comprising test results from microbiology tests performed on microbiology test samples collected using said collection method;
f. generating an indicator of true positive probability based on said contamination rate;
g. using said identifier of said collection method to classify said microbiology test result into said group of test results;
h. generating a report comprising said microbiology test result;
i. and providing said indicator of true positive probability.

2) The system according to claim 1 wherein said specimen vessel is a blood culture bottle.

3) The system according to claim 1 wherein said specimen vessel is a urine specimen container.

4) The system according to claim 1 wherein said indicator of true positive probability is a positive predictive value.

5) The system according to claim 1 wherein said indicator of true positive probability is a descriptive value.

6) The system according to claim 1 wherein said recordkeeping system uses at least one manual recordkeeping process.

7) The system according to claim 1 wherein said recordkeeping system is an electronic medical records system.

8) The system according to claim 1 wherein said identifier of said collection method is further defined as comprised of a decal.

9) The system according to claim 1 wherein said identifier of said collection method is further defined as comprised of an electronic indicator.

10) The system according to claim 9 wherein said electronic indicator is further defined as a field in an electronic recordkeeping system.

11) A system for providing an early indicator of true positive probability of a microbiology test sample comprising the steps of:
a. providing a body fluid vessel comprising a seal having a contact area and a hollow body;
b. providing a specimen vessel comprising a seal and a hollow body;
c. providing a body fluid access system comprising a first end and a second end;
d. providing an identifier of a collection method;
e. drawing a microbiology test sample using said collection method comprising the steps of
i. collecting a body fluid sample by piercing through a patient's skin using said first end of said body fluid access system and piercing through said seal of said body fluid vessel using said second end of said body fluid access system such that potential contaminants are drawn into said body fluid vessel;
ii. collecting a microbiology test sample into said specimen vessel by piercing though said seal of said specimen vessel using said second end of said body fluid access system;
f. performing a microbiology test using said microbiology test sample to yield a microbiology test result;
g. entering said microbiology test result into said recordkeeping system;
h. calculating a contamination rate for a group of test results comprising test results from microbiology tests performed on microbiology test samples collected using said collection method;
i. generating an indicator of true positive probability based on said contamination rate;
j. using said identifier to classify said microbiology test result into said group of test results;
k. generating a report comprising said microbiology test result;
l. and providing said indicator of true positive probability.

12) The system according to claim 11 wherein said identifier of said collection method is attached to said specimen vessel.

13) The system according to claim 11 wherein said identifier of said collection method is further defined as comprised of an electronic indicator.

14) The system according to claim 13 wherein said electronic indicator is further defined as a field in an electronic recordkeeping system.

15) A system for providing an early indicator of true positive probability of a microbiology test sample comprising the steps of:
a. providing a collection device comprising a first end, a second end, and a chamber;
b. providing a specimen vessel;
c. providing an identifier of a collection method;
d. drawing a microbiology test sample using said collection method comprising the steps of
i. collecting a body fluid sample into said collection device through said first end such that potential contaminants are collected into said chamber,
ii. collecting a microbiology test sample through said collection device into said specimen vessel through said second end;
e. performing a microbiology test using said microbiology test sample to yield a microbiology test result;
f. entering said microbiology test result into said recordkeeping system;
g. calculating a contamination rate for a group of test results comprising test results from microbiology tests performed on microbiology test samples collected using said collection method;
h. generating an indicator of true positive probability based on said contamination rate;
i. using said identifier of said collection method to classify said microbiology test result into said group of test results;
j. generating a report comprising said microbiology test result
k. and providing said indicator of true positive probability.

16) The system according to claim 15 wherein said first end of said collection device further comprises a needle.

17) The system according to claim 15 wherein said second end of said collection device further comprises a needle.

18) The system according to claim 15 wherein said identifier of said collection method is further defined as comprised of a decal adhered to said specimen vessel.

19) The system according to claim 15 wherein said identifier of said collection method is further defined as comprised of an electronic indicator.

20) The system according to claim 19 wherein said electronic indicator is further defined as a field in an electronic recordkeeping system.
